(19) United States
(12) Patent Application Publication
(10) Pub. No.: US 2021/0318283 A1
(43) Pub. Date: Oct. 14, 2021

(54) SYSTEMS AND METHODS FOR PROVIDING GERMPLASM CROP SCENARIOS

(71) Applicant: Object Computing, Inc., St. Louis, MO (US)

(72) Inventors: Jason Kendrick Bull, Wildwood, MO (US); Xiao Yang, Chesterfield, MO (US); Allan Francis Trapp, II, St. Louis, MO (US); Tonya Sue Ehlmann, St. Peters, MO (US); Kyu S. Cho, Ballwin, MO (US); James Clesie Moore, III, St. Louis, MO (US); Mohammad Mehdi Kafashan, Wildwood, MO (US)

(21) Appl. No.: 16/845,951

(22) Filed: Apr. 10, 2020

(51) Int. Cl. G01N 33/00; G06N 20/00; G06N 3/08; G01W 1/10; G01N 33/24

(52) U.S. Cl. CPC G01N 33/0098; G06N 20/00; G01N 2033/245; G01W 1/10; G01N 33/246; G06N 3/08

(57) ABSTRACT

Systems and methods are disclosed herein for providing germplasm crop scenarios. The system may calculate a relative maturity (RM) for a germplasm for an area of a location. The system may use weather information associated with a selected area to calculate the relative maturity. The system may then calculate a predictive yield for the germplasm for the area based on the respective relative maturity for the germplasm. The system may then generate the germplasm information for the germplasm indicative of a respective performance for the area of the location based on the respective predictive yield. For example, a plurality of crop scenarios may be generated by date for acorn hybrid seed that provides a more accurate yield calculation based on what date the crop is planted.

### BACKGROUND

The present disclosure is directed to systems and methods for providing predictive models and optimizations for generating germplasm crop scenarios.

### SUMMARY

Achieving successful harvest based on a germplasm may be contingent upon a multitude of factors. In particular, germplasm selection, planting date of the germplasm, and planting location of the germplasm are some of the determinations required for successful harvest. In one approach, yields for germplasms are static and are determined based on statistical yield data. For example, a specific germplasm may be rated to produce a specific amount of yield. However, if the actual field conditions do not match the conditions used to generate the rated yield for the specific germplasm, it is unlikely that the germplasm will meet the rated yield expectations. Determining accurate models for germplasm crop scenarios using statistical yield data remains challenging as the statistical yield data fails to consider required constraints (e.g., specific parameters from grower, geographical characteristics, soil composition, dynamic and historical weather characteristics etc.), required to determine germplasm crop scenarios.

In another approach, germplasm parameters are generated based on historical information for the germplasm without consideration of the contextual environment. As mentioned earlier, this will result in imprecise crop scenarios for the specific germplasm. This problem is exacerbated when comparative germplasms are required for the crop scenario as the aggregate errors are embedded in the derived results. Any comparison of the particular germplasm to other germplasms fails to include contextual information for the particular germplasm in the proposed environment.

Accordingly, techniques are disclosed herein for providing germplasm crop scenarios. The system may calculate a relative maturity (RM) for a germplasm for an area of a location. For example, a relative maturity metric (e.g., the number of days it takes the germplasm to grow to harvest) may be determined for a corn hybrid seed in particular acreage in Fresno Calif. The system may use weather information associated with the acreage in Fresno California to calculate the relative maturity. The system may then calculate a predictive yield for the germplasm for the area at the location based on the respective relative maturity for the germplasm. The system may then generate the germplasm information for the germplasm indicative of a respective performance for the area of the location based on the respective predictive yield. For example, a plurality of crop scenarios may be generated by planting date for the corn hybrid seed that provides a more accurate yield calculation.

In some embodiments, the system calculates the predictive yield based on year to year variance data associated with the germplasm, a product ranking of the germplasm, or penalty data associated with the germplasm. In some embodiments, penalty data may be based on historical moisture data. For example, if the germplasm has excess moisture greater than a predetermined threshold, a penalty value may be determined for the germplasm based on the amount of excess moisture. In some embodiments, the system calculates the predictive yield based on year to year disease variance data associated with germplasm. The year to year disease variance data may further be associated with the respective location.

In some embodiments, the system calculates the relative maturity based on determining, for each acre of the area of the location, an aggregate Growth Degree Days (GDD) value based on historical weather information associated with the location. The system may modify the GDD by statistical operations and use the modified GDD to calculate relative maturity.

In some embodiments, the system implements a machine learning model via control circuitry to determine the germplasm information (e.g., crop scenarios) of the germplasm, the relative maturity of the germplasm, and/or the predictive yield of the germplasm. In some embodiments, the machine learning model may be a neural network with training data based on year to year variance data associated with the germplasm, locational weather information and/or weather volatility value predictive of a likelihood of weather prediction error, and penalty data associated with the germplasm.

### BRIEF DESCRIPTION OF THE DRAWINGS

The below and other objects and advantages of the disclosure will be apparent upon consideration of the following detailed description, taken in conjunction with the accompanying drawings, in which like reference characters refer to like parts throughout, and in which:

FIG. 1 shows an illustrative diagram of a plurality of modules for providing germplasm crop scenarios, in accordance with some embodiments of the disclosure;

FIG. 2A shows an illustrative diagram of a performance prediction engine, constraint engine, weather engine, and germplasm scenarios, in accordance with some embodiments of the disclosure;

FIG. 2B shows exemplary performance variance for germplasms, in accordance with some embodiments of the disclosure;

FIG. 3 shows an illustrative diagram of relative maturity zones for a particular germplasm, in accordance with some embodiments of the disclosure;

FIG. 4 shows an illustrative diagram for determining a relative maturity acre metric for a germplasm, in accordance with some embodiments of the disclosure;

FIG. 5A shows an illustrative diagram depicting harvest moisture correlated with product relative maturity, in accordance with some embodiments of the disclosure;

FIG. 5B shows an illustrative diagram for determining a yield penalty for a germplasm, in accordance with some embodiments of the disclosure;

FIG. 5C shows an illustrative diagram depicting penalty functions by zone for a germplasm, in accordance with some embodiments of the disclosure;

FIG. 6 shows an illustrative diagram for determining a performance index for germplasms, in accordance with some embodiments of the disclosure;

FIG. 7 shows an illustrative diagram for determining predictive yields for germplasms, in accordance with some embodiments of the disclosure;

FIG. 8 shows an illustrative diagram of predicted planting dates for germplasms, in accordance with some embodiments of the disclosure;

FIG. 9 shows an illustrative diagram of performing statistical operations on data from a weather-based relative maturity matrix, in accordance with some embodiments of the disclosure;

FIG. 10 shows an illustrative diagram of germplasm performance distribution over different weather scenarios, in accordance with some embodiments of the disclosure;

FIG. 11A shows an illustrative diagram of determining comparative germplasms based on the constraint engine, in accordance with some embodiments of the disclosure;

FIG. 11B shows an illustrative diagram of determining a sorted list of germplasm scenarios based on the constraint engine, in accordance with some embodiments of the disclosure;

FIG. 12 shows an illustrative block diagram of the modelling engine, in accordance with some embodiments of the disclosure;

FIG. 13 provides an example diagram illustrating the process of training and generating system modules via an artificial neural network, in accordance with some embodiments of the disclosure;

FIG. 14 is an illustrative flowchart of a process for providing germplasm information for a location, in accordance with some embodiments of the disclosure; and

FIG. 15 is an illustrative flowchart of a process for providing germplasm information, for each bucket, for a location, in accordance with some embodiments of the disclosure.

### DETAILED DESCRIPTION

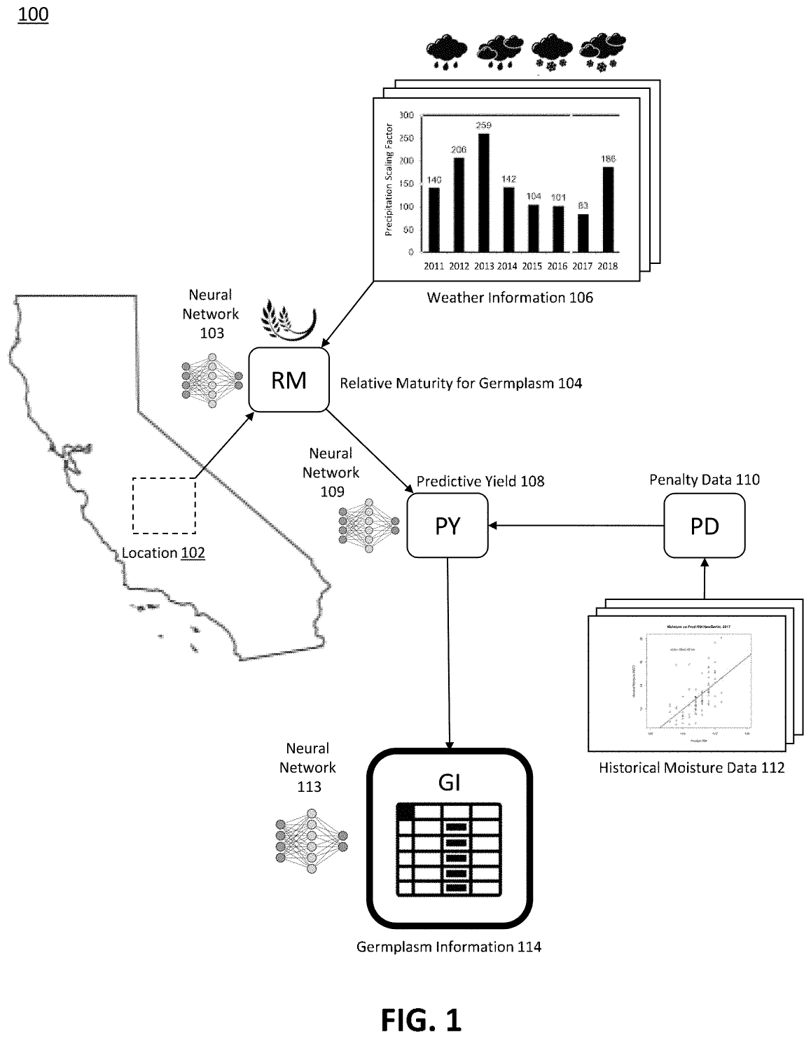

FIG. 1 shows an illustrative diagram 100 of a plurality of modules for providing germplasm crop scenarios, in accordance with some embodiments of the disclosure. A modelling engine may be implemented to provide germplasm crop scenarios. In some embodiments, the modelling engine includes control circuitry to implement the computer system modelling for provision of germplasm crop scenarios. The modelling engine may determine a location. A location may be any parcel of land or water based on a coordinate system (e.g., latitude and/or longitude). Exemplary locations may include, but are not limited to, subdivisions, towns, cities, states, countries, geographic area(s) having a common characteristic(s), geographic areas by defined coordinates, etc. In some embodiments, the modelling engine receives input for the specific location from a user device (e.g., via a communications message and/or a user interface input selection). For example, a location determined by the modelling engine may be the city Fresno located in the state of California, United States of America. The modelling engine may further determine an area within the location. The specific area within the location may be of any level of measurable granularity for area. Exemplary areas may include, acres of a location, square meters of a location, area within the location which may have agricultural utility, defined geographic areas by coordinates, etc. In FIG. 1, the location 102 is Fresno Calif., and the specific area is a farm within Fresno covering 20 acres.

The modelling engine may calculate a relative maturity for one or more germplasms for the area of the location based on weather information associated with the location. A germplasm may be one or more germ cells, a crop, hybrid crop, and/or similar type of biological matter or product. Relative maturity may be the thermal time between planting and physiological maturity of the germplasm. The modelling engine may receive weather information that is used to calculate the relative maturity for the germplasm. The weather information may be any information or dataset having thermal or weather phenomena-based characteristics. For example, weather information may include historical weather information for the specific area and/or location (e.g., 30+ year precipitation patterns for the area). Exemplary weather information may include, but is not limited to, precipitation information, wind speed information, humidity information, air pressure information, moisture information, thermal information, region weather trends, weather modelling for the area. In some embodiments, the weather information may be based on historical weather information for the area or location. In some embodiments, the weather information may be based on simulated weather information for the area or location. In some embodiments, the weather information may include hypothetical modelling for specific scenarios (e.g., drought, floods, tornado, hurricane, infestation of insects, etc.). In some embodiments, there may be a temporal component to the weather information. Returning to FIG. 1, the relative maturity of the germplasm 104 is based on weather information 106 and inserted into a neural network 103. The weather information in this example provides for a precipitation scaling factor for the years 2011-2018. The precipitation scaling factor may be derived based on the output of statistical formulas applied to raw precipitation information for years 2011-2018.

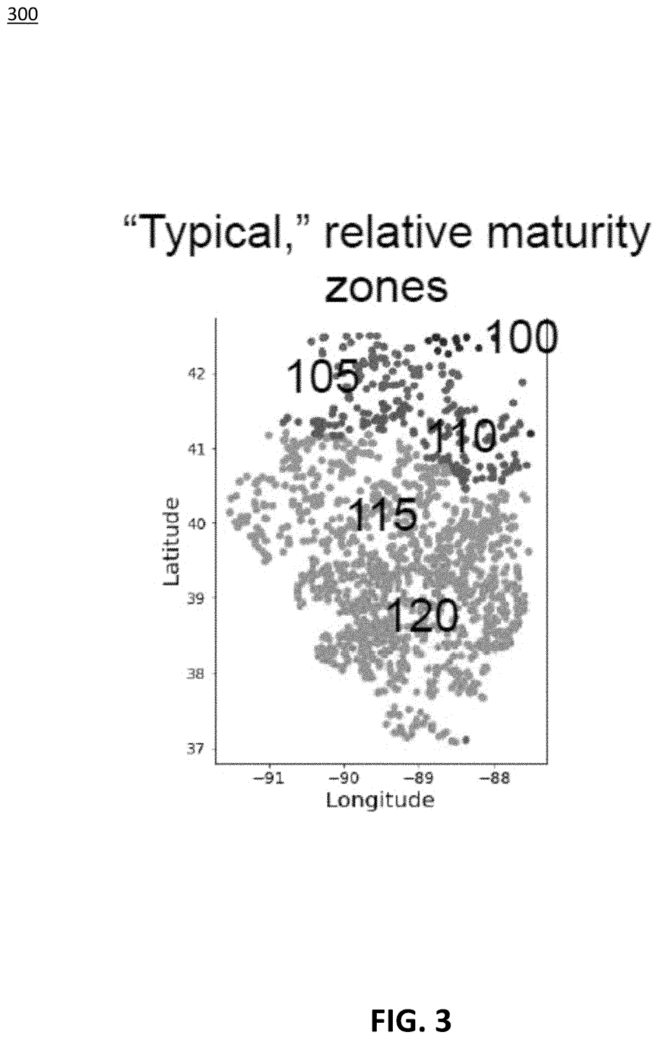

FIG. 3 shows an illustrative diagram 300 of relative maturity zones for a particular germplasm, in accordance with some embodiments of the disclosure. The figure maps latitude to longitude to show the relative maturity values plotted. For example, the north west of the graph (e.g., approximately longitude 88 and latitude 42) indicates a relative maturity value of 100.

The modelling engine may calculate the relative maturity based on a variety of calculations. In some embodiments, the modelling engine receives a “product relative maturity” for a germplasm. The product relative maturity may be derived by the manufacturer of the germplasm putting a static relative maturity value that may not be predictive of the specific relative maturity in a particular environment. It may be an averaged product relative maturity to ensure a baseline accuracy. In this embodiment, the modelling engine may calculate relative maturity by applying statistical operations to the product relative maturity and weather information to result in a relative maturity which is corrected for area specific weather information. The specific type of statistical operations performed may include the calculation of mean and variance geospatially and temporally. In some embodiments, the modelling engine may implement machine learning to determine the relative maturity. In some embodiments, the machine learning may include a neural network (e.g., a convolutional neural network). The neural network may be trained with weather information for the area (e.g., historical weather information and/or simulation weather information for the area). The neural network receives as input the current weather information for the area and the product relative maturity. The neural network would output the relative maturity based on the trained dataset selecting, for example, the most probable weather patterns over the next growing cycle for the germplasm.

In some embodiments, the modelling engine receives additional information for calculating the relative maturity. For example, the modelling engine may receive soil characteristics, pesticide and herbicide characteristics, and/or other location or area-based characteristics for determining relative maturity. This additional information may be applied to one or more statistical operations prior to being input into the relative maturity calculation by the modelling engine. For example, the statistical operations may include, but are not limited to, geospatial smoothing of such soil characteristics like CEC (“Cation-Exchange Capacity”), organic matter, texture, water holding capacity.

In some embodiments, the modelling engine, when calculating the relative maturity of the germplasm, determines for each acre of the area of the location, an accumulated growth degree days (“aGDD”) value based on historical weather information associated with the location. A GDD may be a quantitative value(s) used to describe thermal time where values represent the amount of heat accumulated over a period of time for the germplasm.

The modelling engine may calculate a predictive yield for at least some of the germplasms for the area based on the respective relative maturity for the germplasms. Yield may be the amount of harvested germplasm per land unit (e.g., if the germplasm is corn, a yield may be 200 bushels per acre). Yield may be a ratio of germplasm seeds to output harvested germplasm (e.g., if the germplasm is corn, and three hundred grains of corn are harvested for every corn seed planted, the yield is 300:1). Predictive yield may be calculated based on a number of methodologies disclosed herein. In some embodiments, the modelling engine may calculate predictive yield based on the relative maturity of the germplasm. The modelling engine may subject the relative maturity values to statistical operations. In some embodiments, the modelling engine may be adjusted by the average performance (e.g., yield or other similar metric) of a plurality of germplasms such that the predictive yield may be relative.

In some embodiments, the modelling engine may calculate predictive yield by implementing machine learning. Returning to FIG. 1, the predictive yield 108 is calculated using a neural network 109 and after receiving the relative maturity for germplasm 104. As an example, the predictive yield by implemented by machine learning may be calculated based on the following formula (other variations to the formula and/or inputs may be altered by a person of ordinary skill in the art):

PredictiveYield_{i,j(k)}=f(δᵢ,τ_{i,j}G×E_{i,j(k)})

The above formula provides for predictive yield for a germplasm “i” in field “k” with simulated environment “j.” The modelling engine may determine a machine learning output, denoted δᵢ, for yield derivation for germplasm i. The modelling engine may determine a machine learning informed yield penalty, denoted τ_{i,j}, for germplasm i in the j simulated environment. The G×E_{i,j(k)} may be the statistical germplasm-by-environment-specific variance. A parameterization of f(δᵢ,τ_{i,j},G×E_{i,j(k)}) may be as follows:

PredictiveYield_{i,j(k)}=f(δᵢ,τ_{i,j},G×E_{i,j(k)})=δᵢ+τ_{i,j}+G×E_{i,j(k)}

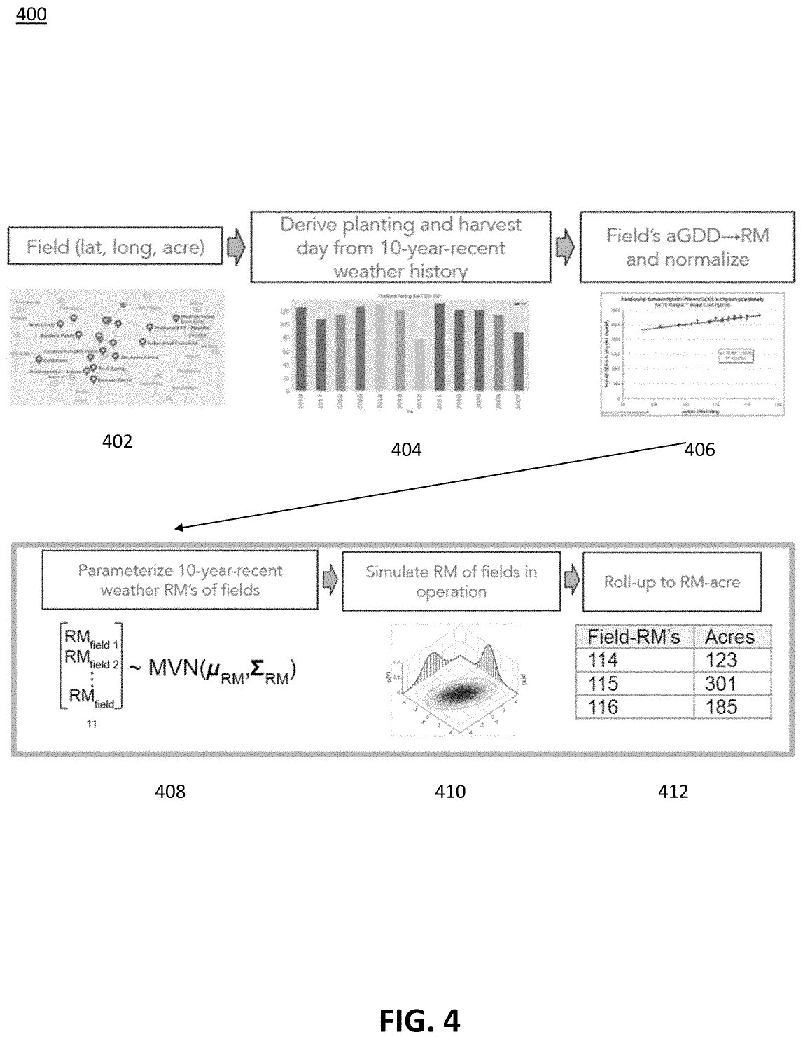

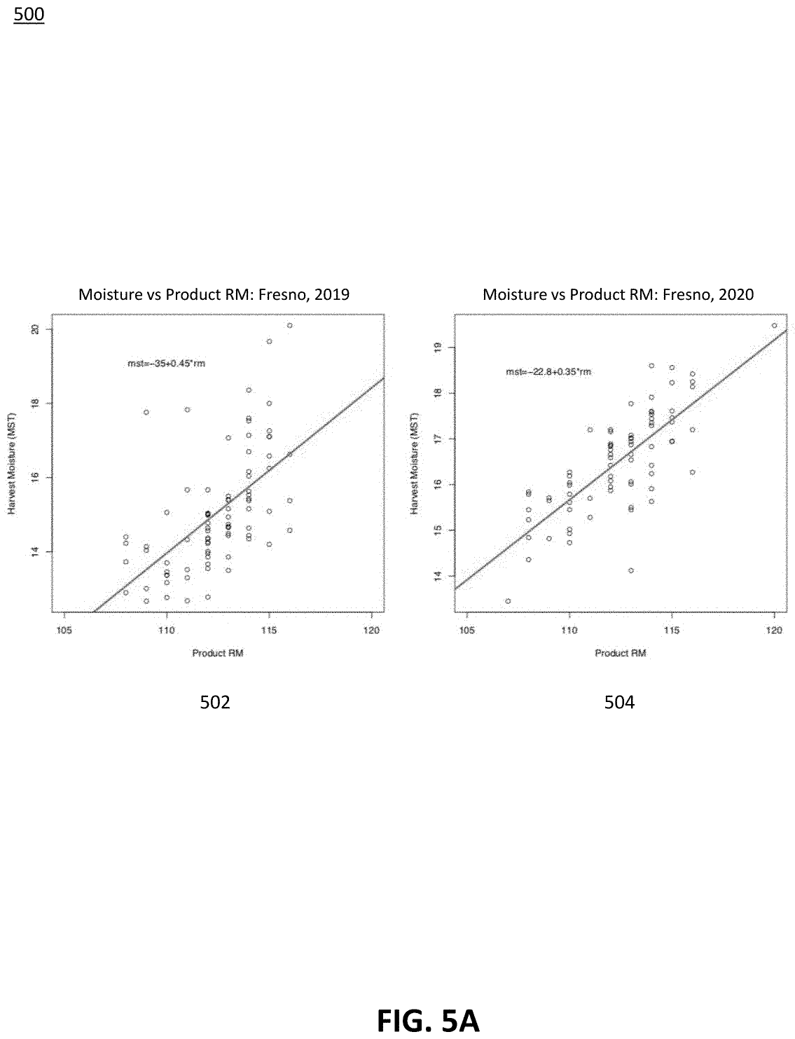

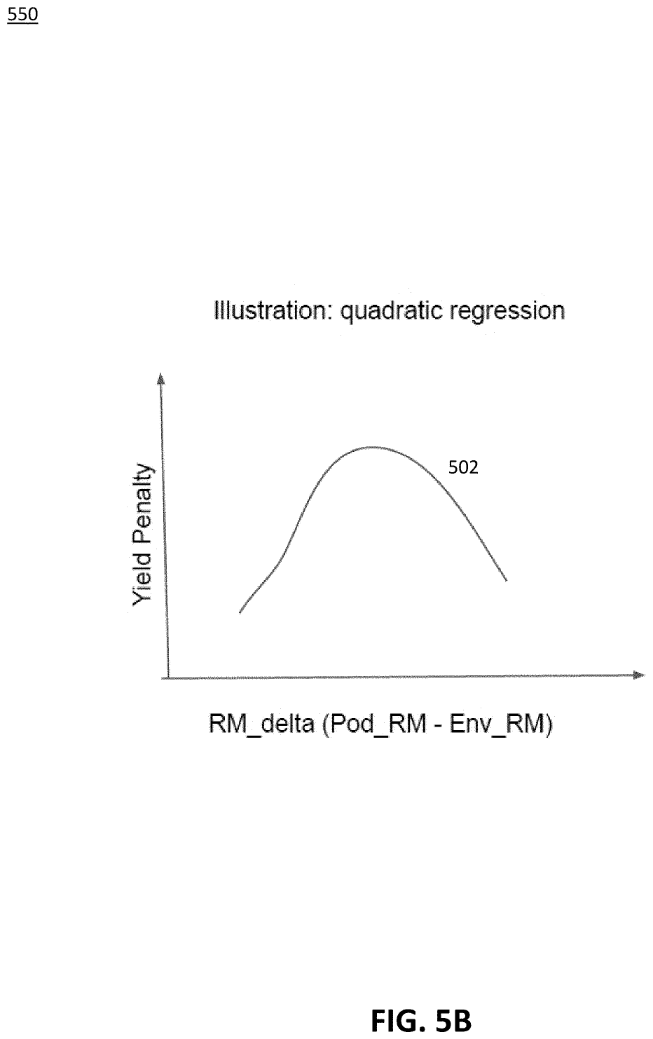

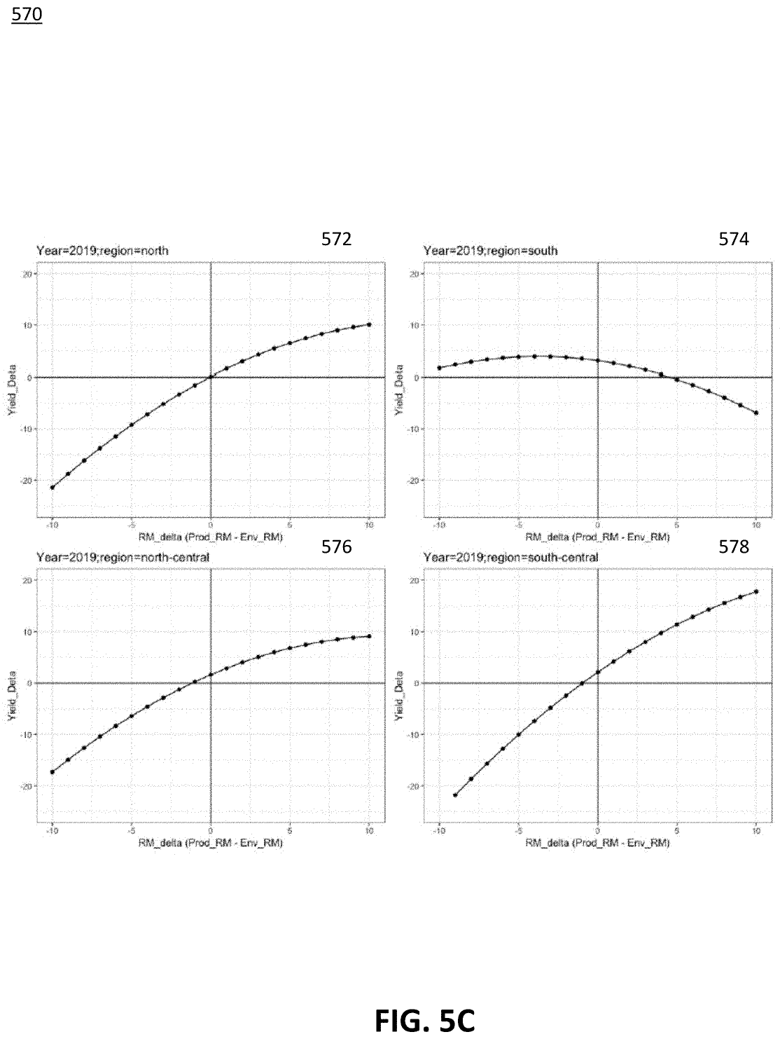

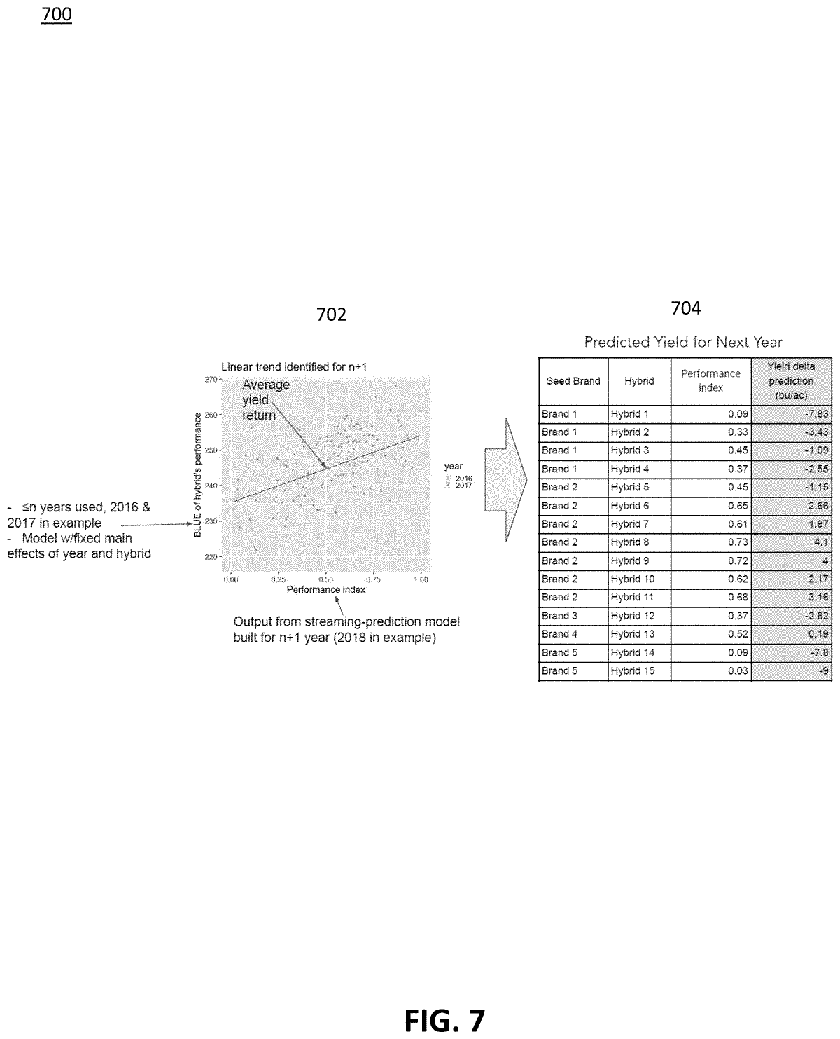

In this equation, δᵢ corresponds to yield delta that may be estimated from a fitting of machine learning outputs. For example, a performance index and/or observed yields may be utilized to determine a fit of the machine learning outputs (e.g., see FIG. 7, specifically 702 illustrating observed yield against performance index). Fitting procedures may range from non-parametric relationships such as locally weighted regression, local polynomial regression, or kernel average smoother to parametric regression such as linear or non-linear regression. Machine learning techniques may include support-vector machines, random forest, and neural networks. The observed yield may be represented as a BLUE, best-linear unbiased estimate of yield, determined through standard analysis of variance, ANOVA, techniques fit to raw yield data. The symbol τ_{i,j} may correspond to the yield penalty that may be the interaction of i^{th} germplasm and j^{th} simulation of environment. This macro parameter may directly account for genetic-by-environment interactions such as hybrid-by-disease, hybrid-by-pest, hybrid-by-weed, hybrid-by-nutrient, hybrid-by-water-availability, and hybrid-by-season-length interactions. For example, plot 502 of FIG. 5A shows how hybrid-by-season-length penalty may be parameterized through a quadratic regression relating yield penalty to the difference of the i^{th} germplasm's relative-maturity and the weighted average of the j^{th} simulation of “relative-maturity-acre.” Relative-maturity-acre may be the collection of fields (e.g., collection of acres), projected to behave with characteristics of the relative maturity of a germplasm. For example, table 412 of FIG. 4 is an iteration of relative-maturity-acre. The relationship of the yield delta penalty to the difference between i^{th} germplasm's relative maturity and the season length of the j^{th} simulation may be a quadratic regression or a non-parametric relationship including locally weighted regression, local polynomial regression, or kernel average smoother. Confounding factors of year and zone may be controlled for by fitting their effects.

G×E_{i,j(k)} may represent the random yield attributed to i^{th} germplasm, in k^{th} field of the j^{th} simulation. This yield term is the remaining ‘noise’ not captured by τ_{i,j} and the δᵢ. It is a general germplasm-by-field-by-year interaction. Yield values may be generated from a statistical distribution, e.g., Gaussian, Weibull, scaled-Gamma, or scaled-Beta, with mean 0 and variance equal to the across-year pooling of hybrid-by-field variances. Hybrid-by-field variances take into account diverse data structures (e.g., randomized-complete block, split-plot, complete-randomized designs, and/or similar structures) and any designed factors (e.g., genetics, pesticide treatments, herbicide treatments, insecticide treatments, nutrient supplementation, seed treatments, and/or any similar factors).

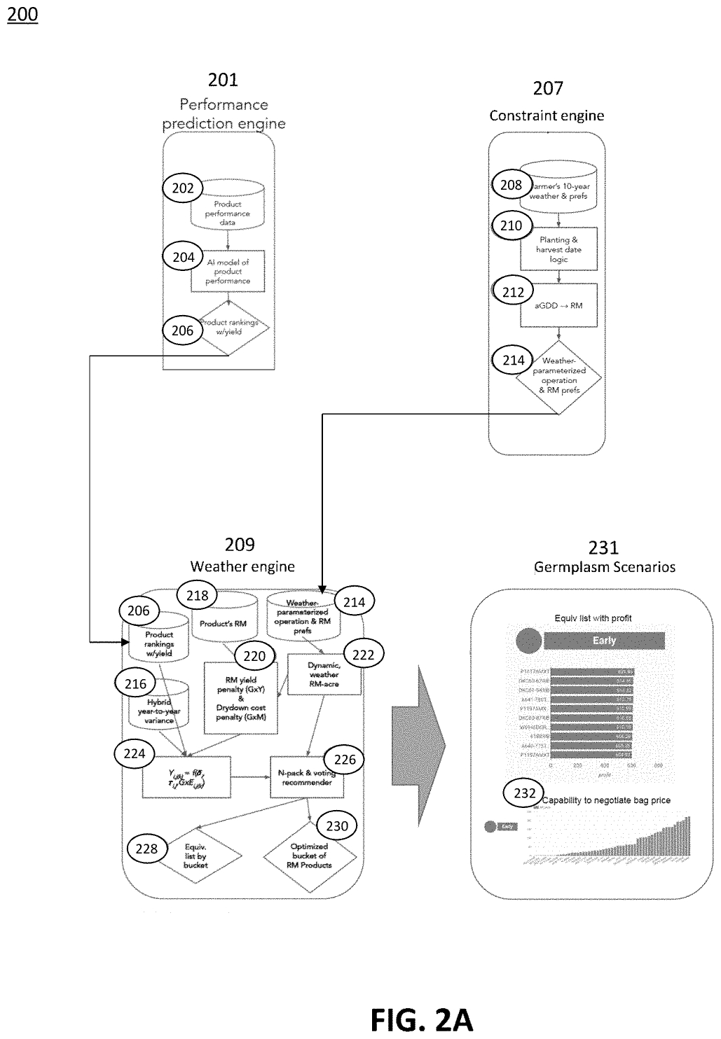

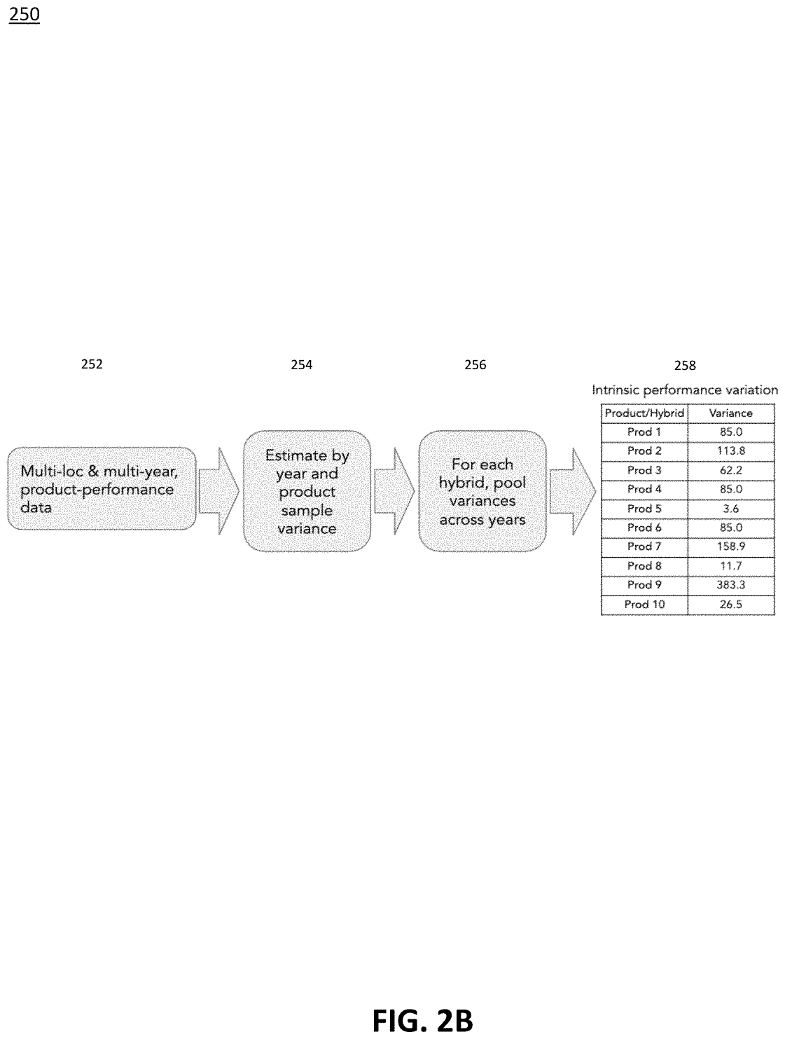

FIG. 2B shows exemplary performance variance for germplasms 250, in accordance with some embodiments of the disclosure. At 252, the modelling engine, through implementation of a machine learning engine, receive training data in form of multi-year product-performance data for a variety of germplasm. This data may be from a system storage or a third-party database. At 254, the modelling engine, through implementation of a machine learning engine, estimates the product sample variance by year for each of the germplasms. At 256, for each germplasm, the modelling engine, through implementation of a machine learning engine, pools the variances across years. The modelling engine may output the intrinsic performance variance for each of the germplasms as shown in 258. This is one exemplary output; however, the output may be in any other format/form which allows for output of identification of the germplasm and the corresponding calculated variance.

In some embodiments, the training of the machine learning model simulates the area for any number of germplasms (i) in and/all areas (j) in a location. For example, the machine learning model may determine predictive yield for every type of germplasm for every acre of a specific farm in Fresno Calif.

FIG. 4 shows an illustrative diagram 400 for determining a relative maturity acre metric for a germplasm, in accordance with some embodiments of the disclosure. At 402, the modelling engine determines the area that may be received in longitude and latitude coordinates. A specific acreage may be extended out as a radius from a singular set of longitude and latitude coordinates. In other embodiments, the specific acreage is provided (e.g., meets and bounds). At 404, the modelling engine derives a relative maturity based on historical weather information (e.g., weather information from the last 10 years). At 406, the modelling engine maps GDD against relative maturity and applies statistical operations to the data. The modelling engine then recursively implements steps 408, 410, and 412. At 408, the modelling engine parameterizes the weather information based on the areas of the relative maturity and simulates, at 410, the relative maturity in their particular areas to derive, at 412, relative maturity acres. In some embodiments, the modelling engine determines, for each acre of the area of the location, an aggregate Growth Degree Days (GDD) value based on historical weather information associated with the location. The modelling engine modifies the aGGD by one or more statistical operations and calculates a relative maturity acreage indicative of the number of acres in the area projected to achieve relative maturity based on the modified aggregate GDD value. In some embodiments, the relative maturity acreage is based on a weather volatility value predictive of a likelihood of weather prediction error. For example, a specific value may be used to indicate likelihood of drought and/or flooding.

In some embodiments, the modelling engine, when calculating predictive yield, may determine penalty data. Penalty data may include a statistical value to adjust the predictive yield if the germplasm matures at a later date than the determined relative maturity. For example, certain germplasms may mature later due to excess moisture. Excess moisture requires dry-down application to remedy the excess moisture. A cost may be associated with the dry-down application to the germplasm. This cost may be used to generate a dry-down cost penalty that may be used to determine the penalty data for the germplasm. Returning to FIG. 1, the predictive yield 108 receives penalty data 110 which is based on historical moisture data 112. FIG. 5A shows an illustrative diagram 500 depicting harvest moisture correlated with product relative maturity, in accordance with some embodiments of the disclosure. At 502, harvest moisture is illustrated on the y-axis while product relative maturity for germplasm named “Fresno” (e.g., the static relative maturity value generally derived from manufacturer) is listed on x-axis for the year 2019. A best fit line is also added for modelling (e.g., best fit line listed as moisture (mst)=35+0.45*(product relative maturity)). In similar fashion, 504 shows the same germplasm Fresno mapped charting harvest moisture against product relative maturity for the year 2020. In some embodiments, the modelling engine may implement a statistical model (random coefficient models) used to generalize relationship between harvest moisture prediction and product relative maturity. For example, moisture may be calculated as per the following formula:

Moisture=a+(b×product_{RM})

In the above equation, a and b are random coefficients across years for a regression model. For example, if the equation is Moisture=35+0.45*product_{RM}, for every unit of product relative maturity increase, approximately 0.5% of Moisture increases (e.g., if product relative maturity increases by 5, then moisture increases 2.5%).

FIG. 5B shows an illustrative diagram for determining a yield penalty 550 for a germplasm, in accordance with some embodiments of the disclosure. A quadratic regression model 502 may be implemented using the following formula:

Δ_{γ}=a+b₁·Δ_{RM}+b₂·Δ²_{RM}

In the above equation, the difference in yield is determined by the coefficient ‘a’ added to coefficient products of b₁ and b₂. The coefficient b₁ is multiplied by the difference in relative maturity for the germplasm (e.g., the difference between product relative maturity and relative maturity [sometime referred to as environmental relative maturity]) and coefficient b₂ is multiplied by the square of the difference in relative maturity for the germplasm. The coefficients (e.g., a, b₁, b₂) may be derived using statistical analysis, or maybe any preset values to initially use the model. In some embodiments, models may be implemented for the quadratic model to explore impact of year and area.

FIG. 5C shows an illustrative diagram 570 depicting penalty functions by zone for a germplasm, in accordance with some embodiments of the disclosure. At 572, an exemplary illustration depicts the difference in yield mapped against the difference in relative maturity for the year 2019 in the north region of the area. At 574, an exemplary illustration depicts the difference in yield mapped against the difference in relative maturity for the year 2019 in the south region of the area. At 576, an exemplary illustration depicts the difference in yield mapped against the difference in relative maturity for the year 2019 in the north-central region of the area. At 578, an exemplary illustration depicts the difference in yield mapped against the difference in relative maturity for the year 2019 in the south-central region of the area.

In some embodiments, the modelling engine, when calculating the predictive yield, may include year to year variance data associated with the germplasm, a product ranking of the germplasm, or penalty data associated with the germplasm. In some embodiments, the year to year variance may include weather information. In some embodiments, the year to year variance may be a metric based on soil composition (e.g., potency of soil composition for the specific germplasm). A product ranking may rank germplasms in order of any desired metric (e.g., highest yield, lowest cost, a preferred ratio of one or more metrics, etc.). In some embodiments, the product ranking may be generated based on at least product relative maturity. In some embodiments, the product ranking may be generated based on at least the relative maturity.

The modelling engine may generate germplasm information for the germplasm indicative of a respective performance for the area of the location based on the respective predictive yield. Germplasm information may be any metric associated with the germplasm. In some embodiments, the germplasm information is a predictive yield value. In some embodiments, the germplasm information may be a relative ranking of a plurality of germplasms based on one or more metrics (e.g., predictive yield, relative maturity, cost per acre, etc.).

In some embodiments, the germplasm information includes a plurality of scenarios for the germplasm. For example, the modelling engine may present an ordered list of a plurality of scenarios in order of respective performance for the particular germplasm. Returning to FIG. 1, the modelling engine, implementing a neural network 113, may generate a spreadsheet of germplasm information 114 including multiple dates for planting and the respective expected relative maturity of the germplasm.

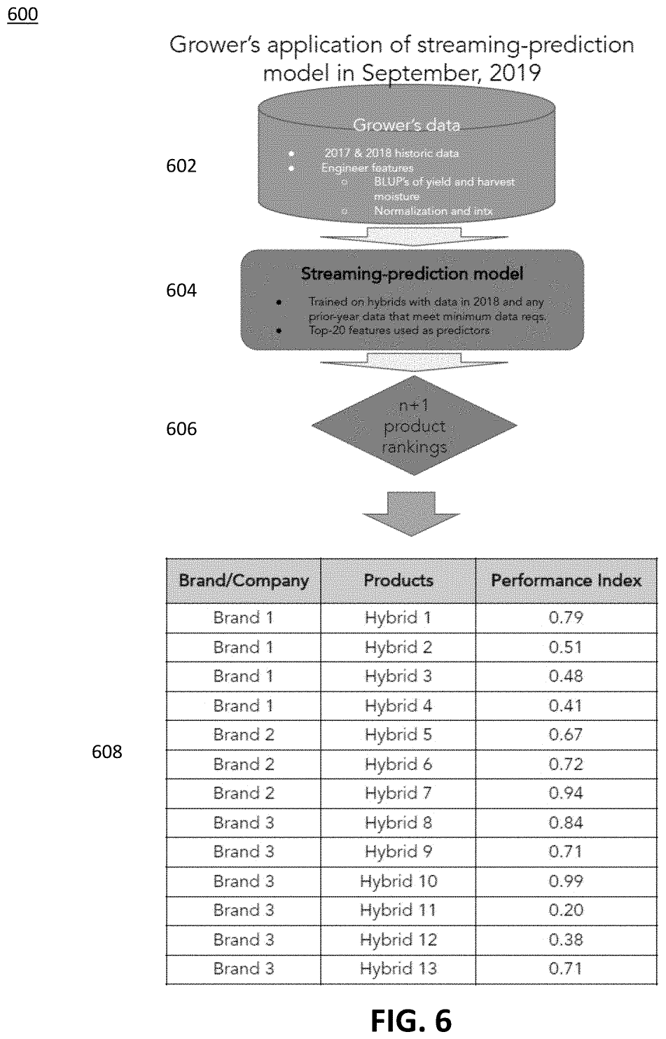

FIG. 6 shows an illustrative diagram 600 for determining a performance index for germplasms, in accordance with some embodiments of the disclosure. The modelling engine may implement a machine learning model to predict an upcoming growing season based on historical data. The machine learning model may be improved as more data becomes available and this new data is added to the training model. A plurality of germplasms may be retrieved for the upcoming growing season and the modelling engine, using the machine learning model, may rank the plurality of germplasms' predictive yield for the upcoming growing season. At 602, the machine learning model may generate specific values for predictive yield and moisture using various statistical operations including, but not limited to, best linear unbiased prediction (“BLUP”), normalization, and other statistical operations. At 604, the machine learning model determines if the generated values are acceptable when compared against respective predefined threshold values. At 606, the machine learning model determines a ranking scheme for each of the plurality of germplasms. In some embodiments, the ranking is based on the previous comparison to predefined threshold values. At 608, the modelling engine generates for output the rankings of the plurality of germplasms. For example, the performance index column may be a specific metric generated for each germplasm. The specific metric may be based on at least one of predictive yield and relative maturity.

FIG. 7 shows an illustrative diagram 700 for determining predictive yields for germplasms, in accordance with some embodiments of the disclosure. In this example, the machine learning model may determine yield delta prediction for each of the plurality of germplasms. At 702, the germplasm (e.g., hybrid) specific performance may be plotted against the performance index values previously generated. The specific performance of the germplasm may be based on best linear unbiased estimator (“BLUE”) methods. The machine learning model may perform a linear regression, or other similar statistical operation, to determine an average yield return model. Upon determining the linear trend, a specific yield delta prediction may be output. The generated output by the machine learning model of the performance index and/or the yield delta prediction for the plurality of germplasms(s) is indicative of germplasm information including a plurality of scenarios.

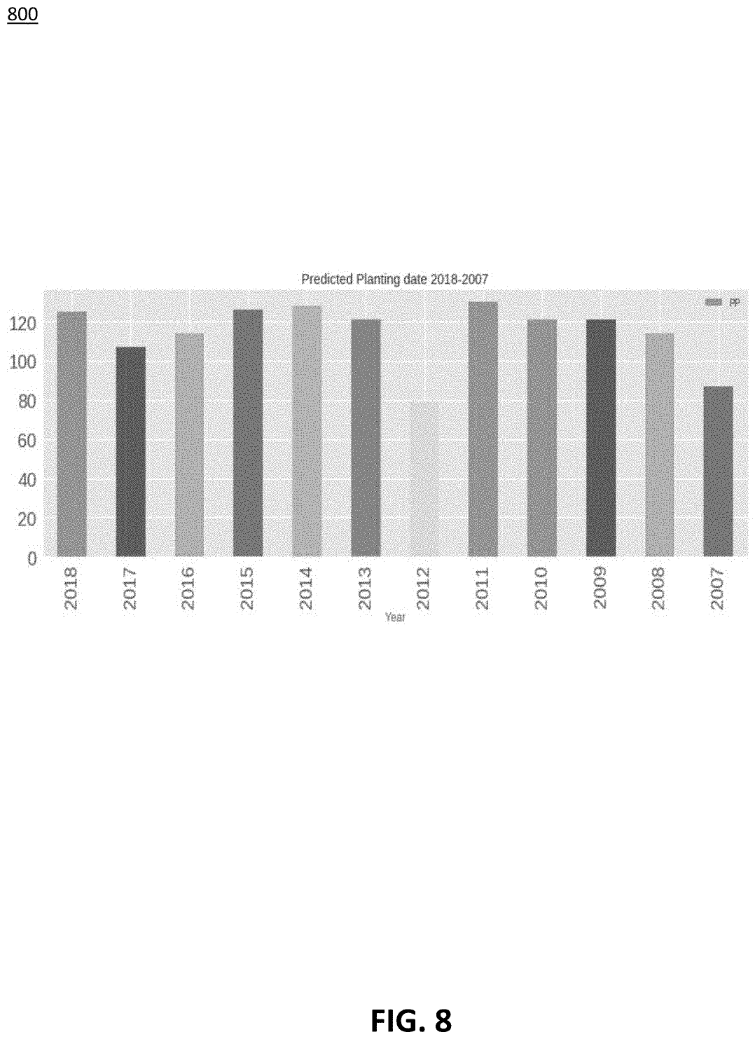

FIG. 8 shows an illustrative diagram 800 of predicted planting dates for germplasms, in accordance with some embodiments of the disclosure. The figure illustrates predictive planting dates for a specific germplasm across years 2007-2018. The y-axis illustrates the amount of days required for harvest. The days may vary year to year based on a variety of factors such as temperature. For example, the machine learning model may use a historical plating day if available. If the historical plating date is not available, the machine learning model may determine a first day of the calendar year where a ten-day average of maximum daily temperature is greater than 63° F. and a seven-day average of daily precipitation is less than 0.8 inches. This determined first day will be used as the plant day for the germplasm.

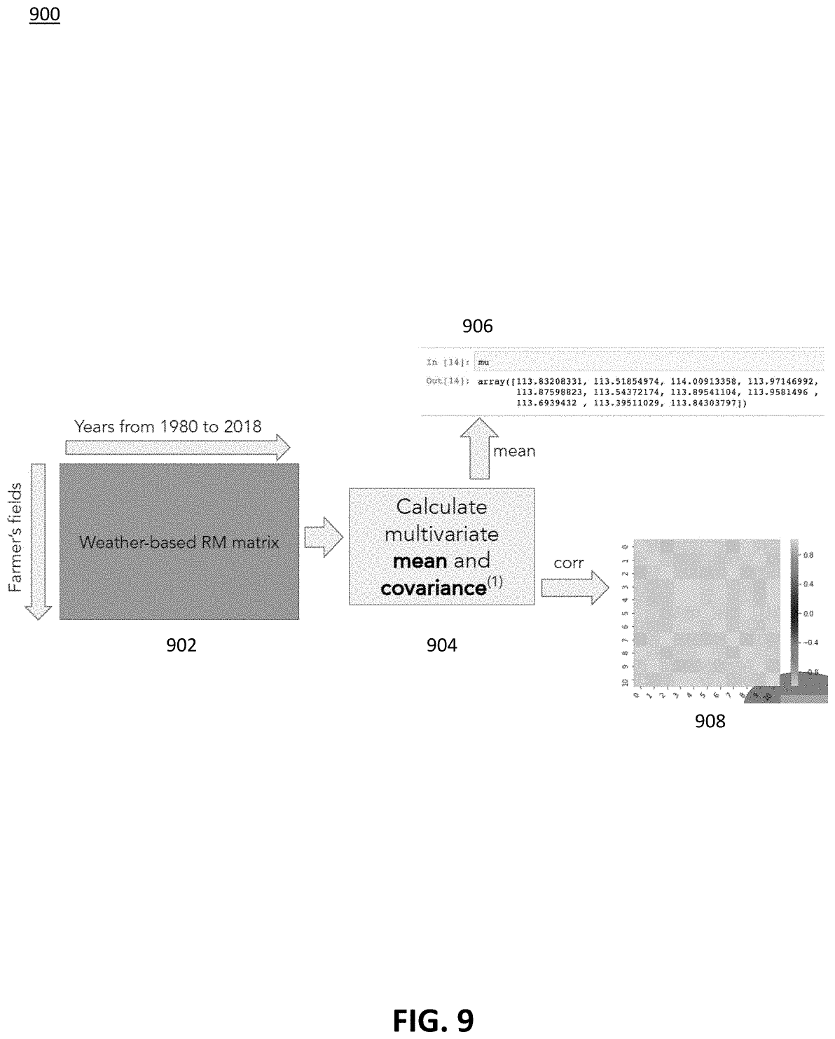

FIG. 9 shows an illustrative diagram 900 of performing statistical operations on data from a weather-based relative maturity matrix, in accordance with some embodiments of the disclosure. In some embodiments, the modelling engine generates a weather-based relative maturity matrix 902 which comprises relative maturity values. The matrix may comprise values based on a timescale (e.g., years such as 1980-2018) and location (e.g., a farmer's specific acreage). The modelling engine may then calculate one or more statistical values 904 from the weather-based relative maturity matrix. For example, multivariate mean and covariance may be calculated. In particular, 906 illustrates an exemplary matrix multivariate mean calculation. In another example, 908 illustrates an exemplary matrix multivariate covariance calculation.

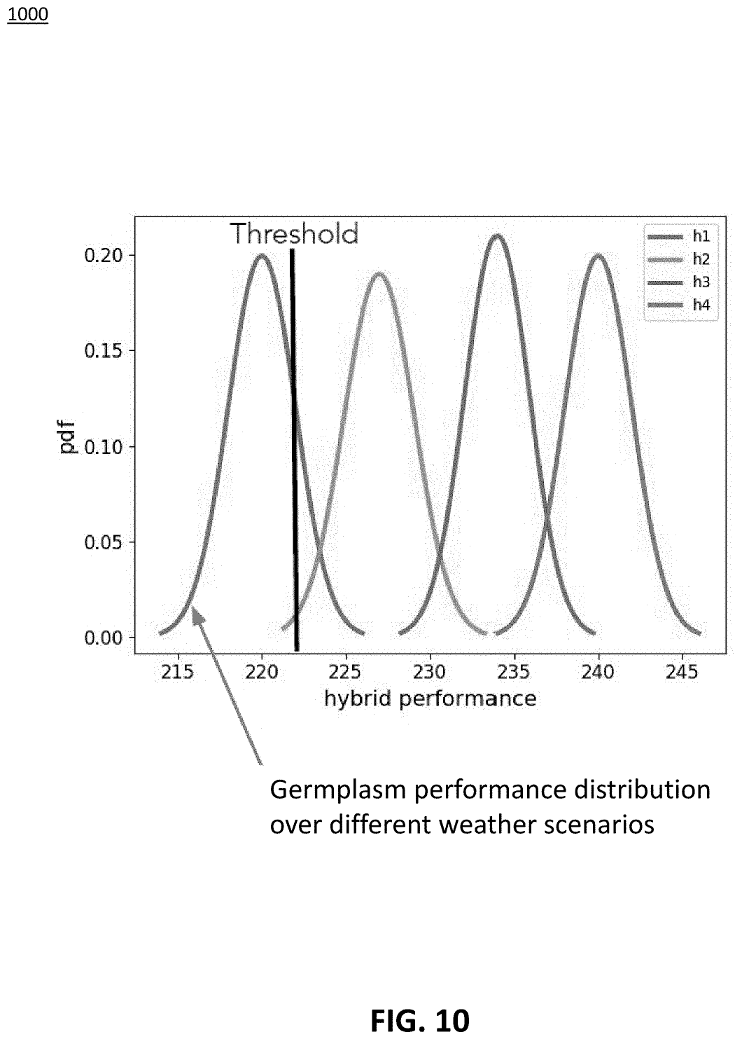

FIG. 10 shows an illustrative diagram 1000 of germplasm performance distribution over different weather scenarios, in accordance with some embodiments of the disclosure. In some embodiments, the modelling engine may group multiple germplasms together to provide comparative analysis of performance. Each germplasm having its own mathematical function represented by the respective bell curves represents germplasm performance distribution over various weather conditions (e.g., the highest value is under optimal weather conditions, while lowest values are under poorest weather conditions). Performance may be measured by a specific performance quantified metric (e.g., probability density function [pdf]) based on one or more inputs such as relative maturity and/or predictive yield. In some embodiments, a threshold may be inserted to provide a probability of a germplasm performance being above a predefined performance threshold. The predefined performance threshold may be based on historical data and/or manufacturer data for the specific germplasm.

In some embodiments, the modelling engine may group a plurality of germplasms in a bucket for comparison. The bucket includes the plurality of germplasms. In some embodiments, the plurality of germplasms for the bucket meets one or more constraint requirements. For example, a constraint may be only related types of germplasms (e.g., specific strains of corn crops). In another example, the constraint may be germplasms which can withstand certain disease. In yet another example, the constraint may be germplasms which may grow in specific locations. In some embodiments, the constraints are automatically generated based on known conditions (e.g., locational information, pest information, previous crop planting patterns, etc.). The processing circuitry may determine the constraints by providing predictive optimization based on historical data. In some embodiments, the modelling engine may determine the constraints using a recommendation engine. For example, the recommendation engine may determine whether one or more germplasms meet key constraints. A germplasm which qualifies under the preset constraints may receive a vote. A germplasm which does not qualify under the preset constraints may receive a risk. The recommendation engine may apply statistical analysis on the votes and risk in aggregate (and/or individually) to determine an “N pack vote” which is the acreage weighted average of yield gain/loss across one or more weather scenarios.

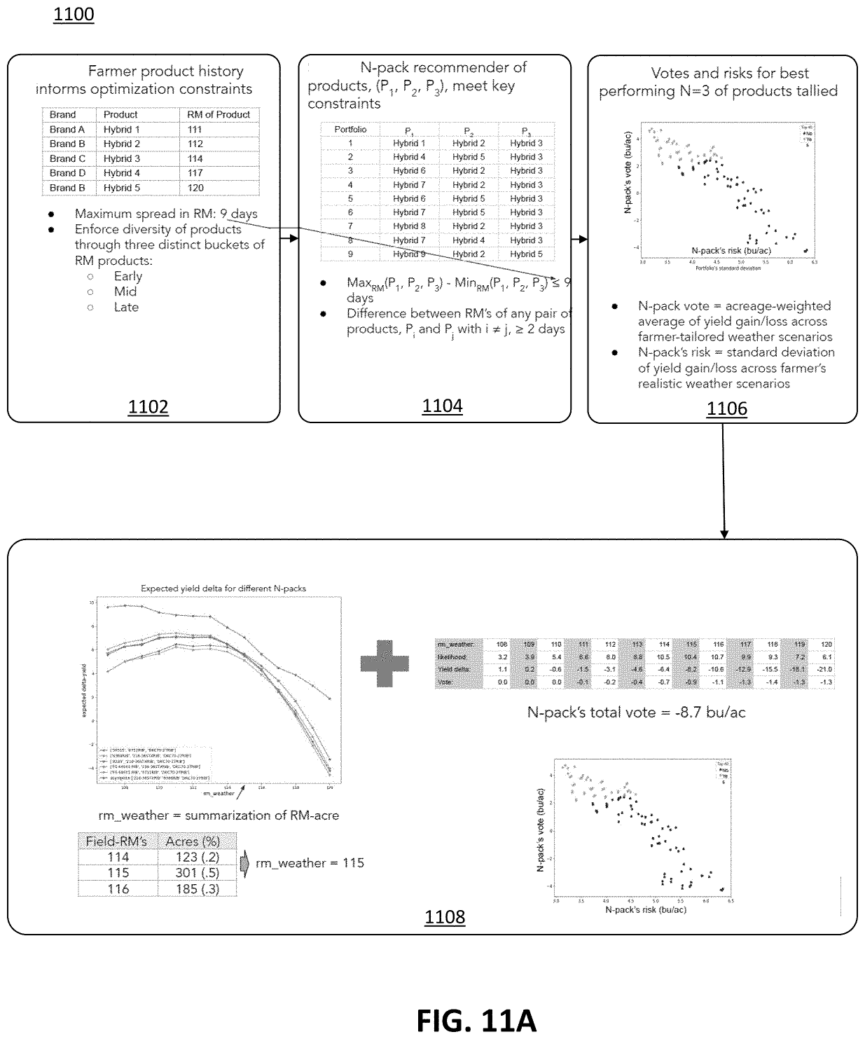

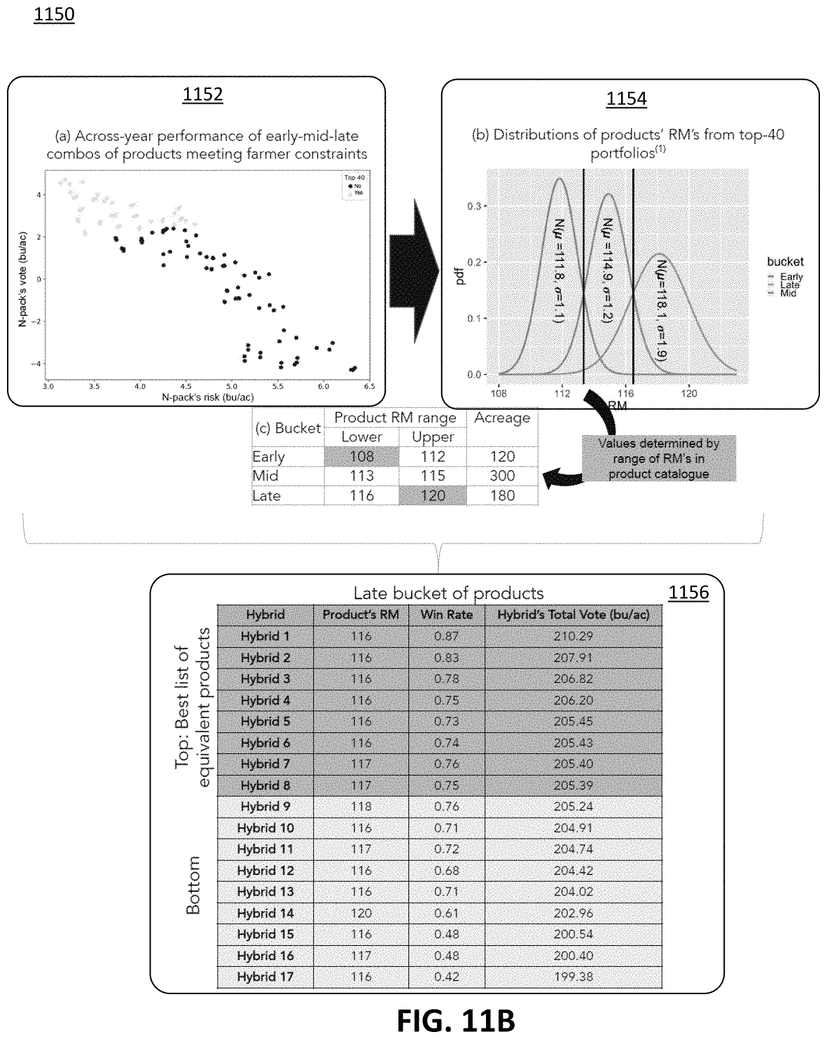

FIG. 11A shows an illustrative diagram 1100 of determining comparative germplasms based on the constraint engine, in accordance with some embodiments of the disclosure. At 1102, the modelling engine may retrieve historical information of a farmer to generate optimization constraints. For example, a constraint regarding the maximum spread of relative maturity for a plurality of germplasms will be set to nine days. Furthermore, the plurality of germplasms may be classified into one of three predefined buckets of relative maturity germplasms (e.g., early RM, middle RM, and late RM).

At 1104, the modelling engine may implement the constraints to generate selection of a subset of germplasms which meet the constraints. In some embodiments, the selection of a subset of germplasms is implemented by a machine learning model. Each of the germplasms are voted by the machine learning model based on whether they satisfy learned conditions. An “N-pack” may be a vote of the (n) germplasms where the pairwise difference in relative maturity constrained by farmer specifications that maximizes yield return under diverse weather scenarios. The N-Pack may be the highest combination of superior products (e.g., germplasms Pₙ) under diverse weather conditions (e.g., the N-Pack may be the result of the vote). The modelling engine may use the following formulas to achieve the example constraints mentioned for germplasm products above:

Max_{RM}(P₁,P₂,P₃)−Min_{RM}(P₁,P₂,P₃)≤9 days

In some embodiments, the difference between the relative maturity of any pair of products Pᵢ and Pⱼ include the following relationship between i and j:

i≠j,≥2 days

At 1106, the modelling engine may determine voting and risk of the germplasm products to determine which of the plurality of germplasm products satisfy the constraints. As shown in 1106, votes are graphed against risk. Each of the units may be of yield per area such as bushels per acre (e.g., bu/ac). Alternatively, metrics of relative maturity acre may be used. In this particular modelling in 1006, the modelling engine, implements various weather scenarios (e.g., variance in temperature, day light hours, humidity, etc.).

At 1108, the modelling engine may determine various metrics for the various germplasm products including predictive yield data.

FIG. 11B shows an illustrative diagram 1500 of determining a sorted list of germplasm scenarios based on the constraint engine, in accordance with some embodiments of the disclosure. The modelling engine, implementing a modelling engine, may output a subset of the germplasms which meet the constraints as shown in 1152 across a year (e.g., growing season). This prediction may also sort, or generate for display, the output of this data with distinction for each of the buckets determined earlier (e.g., early RM, mid RM, and late RM).

At 1154, the modelling engine may determine the distributions from the germplasms which met the constraints for each of the buckets. The predictive yield of each of the plurality of germplasms is mapped against the relative maturity of the germplasms. The modelling engine may also generate this information for display in various output formats (e.g., raw data, charts, graphs, audio summary, video summary, format to use in further analytical applications such as spreadsheet applications).

At 1156, the modelling engine may determine, for a respective bucket, a ranking of all of the germplasm products and various corresponding metrics as scenarios (e.g., germplasm information). For example, a germplasm product' s relative maturity, the win rate (based on statistical operations), and the total vote by the modelling engine may be output for display. The ranking of the germplasm products may be sorted by anyone (or more) of these metrics. The modelling engine may sort the results into tiers such as “Best list of equivalent products” and “bottom” as shown in 1156.

FIG. 2A shows an illustrative diagram 200 of a performance prediction engine, constraint engine, weather engine, and germplasm scenarios, in accordance with some embodiments of the disclosure. The modelling engine may have one of more modules to perform various functions of the aggregate modelling system. In some embodiments, the modelling engine comprises modules including the performance prediction engine 201, constraint engine 207, and weather engine 209. Each of the models provide germplasm scenarios 231.

The performance prediction engine 201 provides for product performance data 202. The product performance data may include metrics associated with the specific germplasm product. For example, a manufacturer labelled product relative maturity. This product relative maturity may not take into consideration the environment for which the product will be harvested. Other product performance data may be included such as an average yield value.

The performance prediction engine 201 provides the product performance data 202 to an AI model of product performance 204. The AI model of product performance 204 utilizing the modelling engine, implementing a machine learning model, to determine specific values such as relative maturity based on environmental location information.

The performance prediction engine 201 provides the AI model of product performance 204 to product rankings with yield 206. The product rankings 206 may be a list of rankings based on product relative maturity or other known product performance data.

The modelling engine may include a weather engine 209 that provides further detailed analysis on germplasm product performance based on weather information. Weather information may be any environmental information for a specific location. For example, the weather information may include precipitation information, wind speed information, humidity information, air pressure information, moisture information, thermal information, region weather trends, weather modelling for the area. The weather engine 209 receives the product rankings with yield 206 and utilizes this information as input for the predictive yield calculation 224. The weather engine may determine the predictive yield calculation by implementing a machine learning model such as a convolutional neural network. The machine learning model may be trained with historical or simulated weather information for a particular area. As mentioned earlier, the predictive yield may be determined based on the following formula:

PredictiveYeild_{i,j(k)}=f(δᵢ,τ_{i,j},G×E_{i,j(k)})

The weather engine receives various inputs to determine the predictive yield including the product rankings with yield 206, the hybrid year-to-year variance 216 (e.g., how much a particular germplasm varies in a metric, such as relative maturity, year after year), the product's relative maturity yield penalty and dry-down cost penalty 220 (based on the product RM 218), and weather information including weather-parameterized operation and PM preferences 214 and dynamic weather RM acre values for specific germplasms 222.

The modelling engine may include a constraint engine 207 which generates constraints to be implemented for the output of germplasm scenarios 231. The constraint engine may receive historical preferences 208 from a particular entity (e.g., farmer, agriculture company, food producer, etc.). For example, historical consumption and behavioral patterns may provide various product preferences, and/or specific practices for fertilization, watering, and/or other practices taken by the entity for specific germplasms. The constraint engine may further receive historical weather information 208 for a particular area. For example, this may include maximum and minimum daily temperatures, relative humidity, daily precipitation accumulated, and windspeed.

The constraint engine may provide the historical preferences and weather information to a planting and harvest date logic module 210. The constraint engine may receive a predetermined planting date. In some embodiments, constraint engine generates a planted date based on the first day of calendar year where 10-day average of maximum daily temperature exceeds 63 degrees Fahrenheit, and 7-day average of daily precipitation is less than 0.8 inches. In some embodiments, if the planting date occurs before April 1, the planting date will automatically be forwarded to April 1. In some embodiments, the constraint engine receives a preset harvest date. In some embodiments, the constraint engine determines the harvest date to be the first date in June where the minimum daily temperature falls below 3 degrees Fahrenheit minus 14 days. The constraint engine may determine a growing degree days (GDD) for a germplasm based on the daily maximum and minimum temperature for a selected location in planting and harvest range. GDD may be determined for a plurality of germplasms and summed. This sum of GDD for the plurality of germplasms may be used to derive a relative maturity of a specific area 212.

The constraint engine may generate parameterized growing season lengths based on constraints and relative maturity preferences 214. For example, the constraint engine may generate a weather-based RM matrix based off the GDD calculations above. The constraint engine may then determine various statistical values by calculating multivariate mean and/or covariance from the weather-based RM matrix. This determined statistical values are forwarded to the weather engine and used in the calculation in the dynamic weather RM-acre 222 (e.g., capturing an entity's most likely weather scenarios).

The weather engine calculates a n-pack (e.g., for the n germplasm products) voting recommendation to determine which of the plurality of germplasm products are within the received constraints 226. The weather engine may list the recommended germplasm products by buckets set by the constraints engine 228. Finally, the weather engine optimizes the buckets based on the RM products which are the highest recommendation Rm products based on the voting recommendation 230.

The weather engine may output the optimized buckets of RM products as list of germplasm scenarios. In the shown figure at 232, a projected profit figure is generated based on the recommended germplasm products for the early RM bucket.

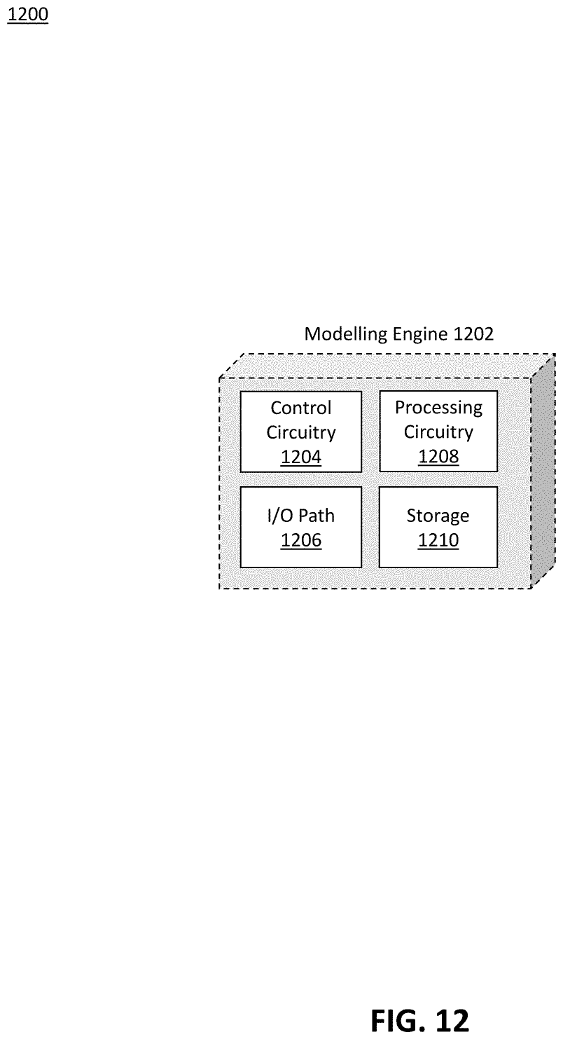

FIG. 12 shows an illustrative block diagram 1200 of the modelling engine, in accordance with some embodiments of the disclosure. In some embodiments, the modelling engine may be communicatively connected to a user interface. In some embodiments, the modelling engine may include processing circuitry, control circuitry, and storage (e.g., RAM, ROM, hard disk, removable disk, etc.). The modelling engine may include an input/output path 1206. I/O path 1206 may provide device information, or other data, over a local area network (LAN) or wide area network (WAN), and/or other content and data to control circuitry 1204, that includes processing circuitry 1208 and storage 1210. Control circuitry 1204 may be used to send and receive commands, requests, signals (digital and analog), and other suitable data using I/O path 1206. I/O path 1206 may connect control circuitry 1204 (and specifically processing circuitry 1208) to one or more communications paths.

Control circuitry 1204 may be based on any suitable processing circuitry such as processing circuitry 1208. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores) or supercomputer. In some embodiments, processing circuitry may be distributed across multiple separate processors or processing units, for example, multiple of the same type of processing units (e.g. two Intel Core i7 processors) or multiple different processors (e.g., an Intel Core i5 processor and an Intel Core i7 processor). In some embodiments, control circuitry 1204 executes instructions for a modelling engine stored in memory (e.g., storage 1210).

Memory may be an electronic storage device provided as storage 410, which is part of control circuitry 1204. As referred to herein, the phrase “electronic storage device” or “storage device” should be understood to mean any device for storing electronic data, computer software, or firmware, such as random-access memory, read-only memory, hard drives, solid state devices, quantum storage devices, or any other suitable fixed or removable storage devices, and/or any combination of the same. Nonvolatile memory may also be used (e.g., to launch a boot-up routine and other instructions).

The modelling engine 1202 may be coupled to a communications network. The communication network may be one or more networks including the Internet, a mobile phone network, mobile voice or data network (e.g., a 5G, 4G or LTE network), mesh network, peer-to-peer network, cable network, or other types of communications network or combinations of communications networks. The modelling engine may be coupled to a secondary communication network (e.g., Bluetooth, Near Field Communication, service provider proprietary networks, or wired connection) to the selected device for generation for playback. Paths may separately or together include one or more communications paths, such as a satellite path, a fiber-optic path, a cable path, a path that supports Internet communications, free-space connections (e.g., for broadcast or other wireless signals), or any other suitable wired or wireless communications path or combination of such paths.

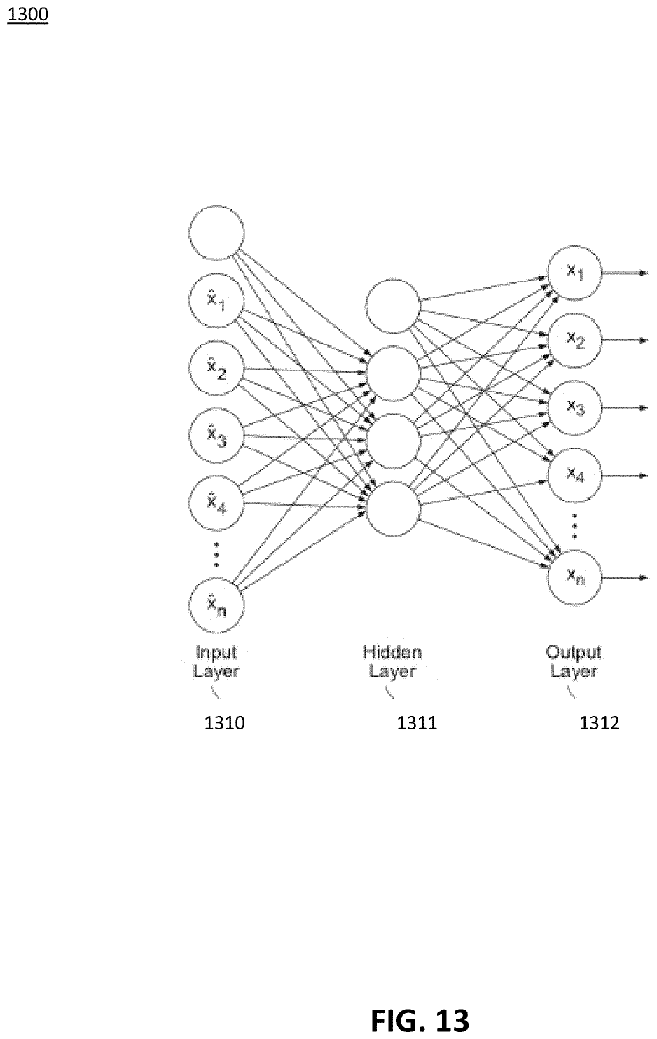

FIG. 13 provides an example diagram 1300 illustrating the process of training and generating system modules via an artificial neural network, in accordance with some embodiments of the disclosure. An artificial neural network may be trained with data based on year to year variance data associated with the germplasm, locational weather information and/or weather volatility value predictive of a likelihood of weather prediction error, and penalty data associated with the germplasm. This data is fed to the input layer 1310 of the artificial neural network. The artificial neural network may be trained to identify the common pattern from different visualizations via processing at one or more hidden layers 1311. Thus, by identifying weather information trends from the input data, predictive weather data is generated at the output layer 1312.

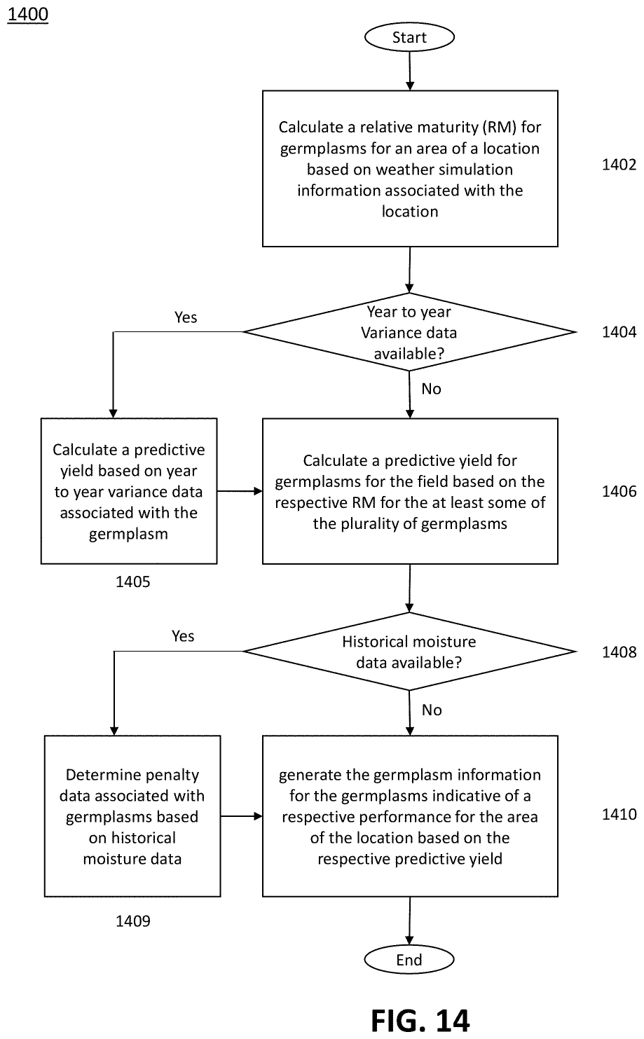

FIG. 14 is an illustrative flowchart of a process 1400 for providing germplasm information for a location, in accordance with some embodiments of the disclosure. Process 700, and any of the following processes, may be executed by control circuitry 1204 of the modelling engine 1202.

At 1402, the modelling engine 1202, by control circuitry 1204, calculates a relative maturity (RM) for germplasms for an area of a location based on weather simulation information associated with the location. In some embodiments, the calculation of relative maturity (RM) for germplasms is calculated, at least in part, by processing circuitry 1208. In some embodiments, the control circuitry 1204 may implement a machine learning model to calculate the relative maturity for germplasms. In some embodiments, at least a portion of the weather simulation information is retrieved from a storage. The storage may be storage 1210 of the modelling engine 1202 or a remote storage (e.g., a database) accessed by the I/O path 1206. In some embodiments, the locational information may be received by the modeling engine 1202 via control circuitry 1204 by an embedded GPS sensor. In some embodiments, the locational information may be received by the modeling engine 1202 via control circuitry 1204 via the I/O path 1206 from a database and/or other electronic device transmitting the locational information to the modeling engine 1202.

At 1404, the modelling engine 1202, by control circuitry 1204, determines whether year to year variance data is available. In some embodiments, the modelling engine 1202 queries an electronic device (e.g., a computer server) or database whether the year to year variance data is available via the I/O path 1206. If, at 1404, control circuitry determines “No,” the year to year variance data is not available, the process advances to 1406.

At 1406, the modelling engine 1202, by control circuitry 1204, calculates a predictive yield for germplasms for the field based on the respective RM for at least some of the plurality of germplasms. In some embodiments, the calculation of predictive yield for germplasms is calculated, at least in part, by processing circuitry 1208. In some embodiments, the control circuitry 1204 may implement a machine learning model to calculate the predictive yield for germplasms.

If, at 1404, control circuitry determines “Yes,” the year to year variance data is available, the process advances to 1405. At 1405, the modelling engine 1202, by control circuitry 1204, calculates a predictive yield based on year to year variance data associated with the germplasm. In some embodiments, the calculation of predictive yield for germplasms is calculated, at least in part, by processing circuitry 1208.

At 1408, the modelling engine 1202, by control circuitry 1204, determines whether historical moisture data is available. In some embodiments, the modelling engine 1202 queries an electronic device (e.g., a computer server) or database whether the moisture data is available via the I/O path 1206. If, at 1404, control circuitry determines “No,” the moisture data is not available, the process advances to 1410.

At 1410, the modelling engine 1202, by control circuitry 1204, generates the germplasm information for the germplasms indicative of a respective performance for the area of the location based on the respective predictive yield. In some embodiments, the generation of germplasm information for germplasms is generated, at least in part, by processing circuitry 1208. In some embodiments, the control circuitry 1204 may implement a machine learning model to generate the germplasm information for the germplasms. In some embodiments, the modelling engine 1202, by control circuitry 1204, may generate for display the generated germplasm information for an electronic device via the I/O path 1206. In some embodiments, the 1202, by control circuitry 1204, may store the germplasm information in storage. The storage may be storage 1210 of the modelling engine 1202 or a remote storage (e.g., a database) accessed by the I/O path 1206.

If, at 1408, control circuitry determines “Yes,” the moisture data is available, the process advances to 1409. At 1409, the modelling engine 1202, by control circuitry 1204, determines penalty data associated with germplasms based on historical moisture data. In some embodiments, the determination of penalty data for germplasms is determined, at least in part, by processing circuitry 1208. In some embodiments, the modelling engine 1202, by control circuitry 1204, receives the moisture data from storage. The storage may be storage 1210 of the modelling engine 1202 or a remote storage (e.g., a database) accessed by the I/O path 1206.

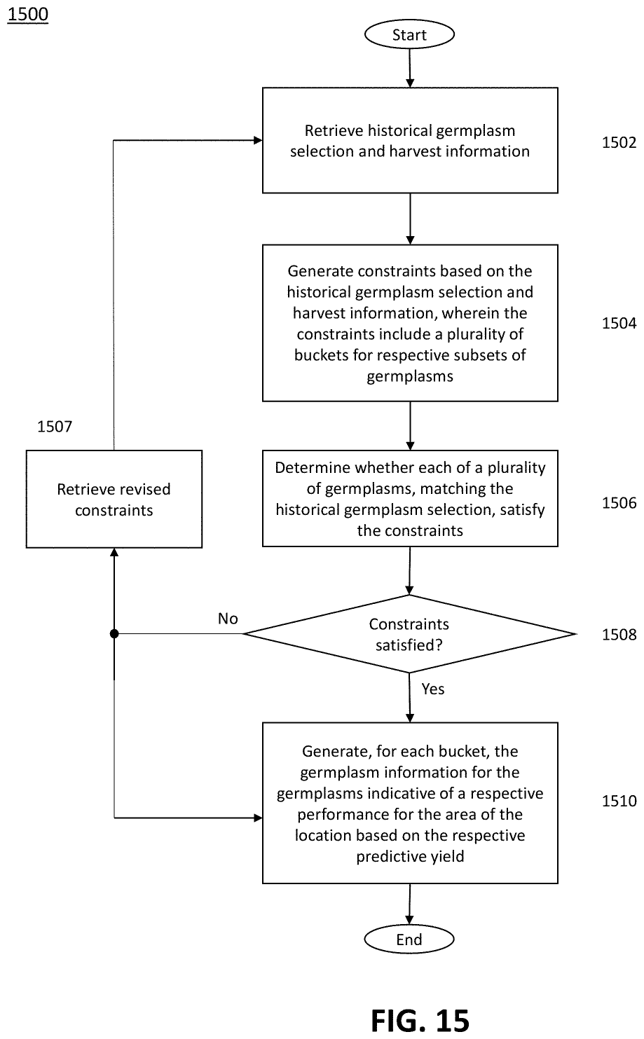

FIG. 15 is an illustrative flowchart of a process 1500 for providing germplasm information, for each bucket, for a location, in accordance with some embodiments of the disclosure. At 1502, the modelling engine 1202, by control circuitry 1204, retrieves historical germplasm selection and harvest information. In some embodiments, the retrieval of historical germplasm selection and harvest information is retrieved from storage. The storage may be storage 1210 of the modelling engine 1202 or a remote storage (e.g., a database) accessed by the I/O path 1206.

At 1504, the modelling engine 1202, by control circuitry 1204, generates constraints based on the historical germplasm selection and harvest information. The constraints include a plurality of buckets for respective subsets of germplasms. In some embodiments, the generation of constraints is generated, at least in part, by processing circuitry 1208. In some embodiments, the modelling engine 1202, by control circuitry 1204, may implement a machine learning model to generate constraints.

At 1506, the modelling engine 1202, by control circuitry 1204, determines whether each of a plurality of germplasms, matching the historical germplasm selection, satisfy the constraints. In some embodiments, the determination of whether each of a plurality of germplasms, matching the historical germplasm selection, satisfy the constraints, is performed, at least in part, by processing circuitry 1208.

At 1508, the modelling engine 1202, by control circuitry 1204, determines whether each of a plurality of germplasms, matching the historical germplasm selection, satisfy the constraints. In some embodiments, if, at 1508, control circuitry determines “No,” at least one of the plurality of germplasms, matching the historical germplasm selection, does not satisfy the constraints, the process advances to 1507. At 1507, the modelling engine 1202, by control circuitry 1204, retrieves revised constraints and reverts to step 1502. The revised constraints may be altered by a pre-configured amount. In some embodiments, if, at 1508, control circuitry determines “No,” at least one of the plurality of germplasms, matching the historical germplasm selection, does not satisfy the constraints, the process advances to 1510.

If, at 1508, control circuitry determines “Yes,” at least one of the plurality of germplasms, matching the historical germplasm selection, satisfies the constraints, the process advances to 1510. At 1510, the modelling engine 1202, by control circuitry 1204, generates, for each bucket, the germplasm information for the germplasms indicative of a respective performance for the area of the location based on the respective predictive yield. In some embodiments, the generation of germplasm information for each bucket is generated, at least in part, by processing circuitry 1208. In some embodiments, the control circuitry 1204 may implement a machine learning model to generate the germplasm information for each bucket. In some embodiments, the modelling engine 1202, by control circuitry 1204, may generate for display the generated germplasm information for each bucket on an electronic device via the I/O path 1206. In some embodiments, the 1202, by control circuitry 1204, may store the germplasm information for each bucket in storage. The storage may be storage 1210 of the modelling engine 1202 or a remote storage (e.g., a database) accessed by the I/O path 1206.

It is contemplated that some suitable steps or suitable descriptions of FIGS. 14-15 may be used with other suitable embodiment of this disclosure. In addition, some suitable steps and descriptions described in relation to FIGS. 14-15 may be implemented in alternative orders or in parallel to further the purposes of this disclosure. For example, some suitable steps may be performed in any order or in parallel or substantially simultaneously to reduce lag or increase the speed of the system or method. Some suitable steps may also be skipped or omitted from the process. Furthermore, it should be noted that some suitable devices or equipment discussed in relation to FIGS. 12-13 could be used to perform one or more of the steps in FIGS. 14-15.

The processes discussed above are intended to be illustrative and not limiting. One skilled in the art would appreciate that the steps of the processes discussed herein may be omitted, modified, combined, and/or rearranged, and any additional steps may be performed without departing from the scope of the invention. More generally, the above disclosure is meant to be exemplary and not limiting. Only the claims that follow are meant to set bounds as to what the present invention includes. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods.

What is claimed is:

1. A method for providing germplasm information for a location, the method comprising:
calculating a relative maturity (RM) for each of a plurality of germplasms for an area of the location based on weather information associated with the location;
calculating a predictive yield for at least some of the plurality of germplasms for the area based on the respective RM for the at least some of the plurality of germplasms; and
generating the germplasm information for the at least some of the plurality of germplasms indicative of a respective performance for the area of the location based on the respective predictive yield.

2. The method of claim 1, wherein calculating the predictive yield comprises calculating the predictive yield based on at least one of year to year variance data associated with the germplasm, a product ranking of the germplasm, or penalty data associated with the germplasm.

3. The method of claim 1 further comprising determining penalty data associated with each of the at least some of the plurality of germplasms based on historical moisture data, wherein calculating the predictive yield comprises calculating the predictive yield further based on the penalty data.

4. The method of claim 1, wherein calculating the RM of each of the germplasms comprises:
determining, for each acre of the area of the location, an aggregate Growth Degree Days (GDD) value based on historical weather information associated with the location;
modifying the aggregate GDD value by one or more statistical operations; and
calculating an RM acreage indicative of the number of acres in the area projected to achieve relative maturity based on the modified aggregate GDD value.

5. The method of claim 4, wherein calculating the RM acreage comprises calculating the RM acreage based on a weather volatility value predictive of a likelihood of weather prediction error.

6. The method of claim 1, wherein the germplasm information comprises a plurality of scenarios, the method further comprising presenting an ordered list of the plurality of scenarios in order of respective performance.

7. The method of claim 1, wherein the calculation of at least one of the RM or the predictive yield are implemented via control circuitry using a machine learning model.

8. The method of claim 7, wherein the machine learning model comprises at least one of: a neural network, a deep neural network, a convolutional neural network, or a generative adversarial network.

9. The method of claim 2, wherein calculating the predictive yield further comprises calculating the predictive yield based on at least one of year to year disease variance data associated with germplasm.

10. The method of claim 9, wherein the disease variance data associated with germplasm is based on locational data.

11. A system for providing germplasm information for a location, the system comprising:
control circuitry configured to:
calculate a relative maturity (RM) for each of a plurality of germplasms for an area of the location based on weather information associated with the location;
calculate a predictive yield for at least some of the plurality of germplasms for the area based on the respective RM for the at least some of the plurality of germplasms; and
generate the germplasm information for the at least some of the plurality of germplasms indicative of a respective performance for the area of the location based on the respective predictive yield.

12. The system of claim 11, wherein the control circuitry is configured, when calculating the predictive yield, to calculate the predictive yield based on at least one of year to year variance data associated with the germplasm, a product ranking of the germplasm, or penalty data associated with the germplasm.

13. The system of claim 11, wherein the control circuitry is further configured to determine penalty data associated with each of the at least some of the plurality of germplasms based on historical moisture data, wherein calculating the predictive yield comprises calculating the predictive yield further based on the penalty data.

14. The system of claim 11, wherein the control circuitry, when calculating the RM of each of the germplasms, to:
determine, for each acre of the area of the location, an aggregate Growth Degree Days (GDD) value based on historical weather information associated with the location;
modify the aggregate GDD value by one or more statistical operations; and
calculate an RM acreage indicative of the number of acres in the area projected to achieve relative maturity based on the modified aggregate GDD value.

15. The system of claim 14, wherein the control circuitry is configured to, when calculating the RM acreage, calculate the RM acreage based on a weather volatility value predictive of a likelihood of weather prediction error.

16. The system of claim 11, wherein the germplasm information comprises a plurality of scenarios, and the control circuitry is further configured to present an ordered list of the plurality of scenarios in order of respective performance.

17. The system of claim 11, wherein the calculation of at least one of the RM or the predictive yield are implemented via control circuitry using a machine learning model.

18. The system of claim 17, wherein the machine learning model comprises at least one of: a neural network, a deep neural network, a convolutional neural network, or a generative adversarial network.

19. The system of claim 12, wherein the control circuitry is configured, when calculating the predictive yield, to further calculate the predictive yield based on at least one of year to year disease variance data associated with germplasm.

20. The system of claim 19, wherein the disease variance data associated with germplasm is based on locational data.

21. A non-transitory computer readable medium having instructions encoded thereon, that when executed by control circuitry causes the control circuitry to:
calculate a relative maturity (RM) for each of a plurality of germplasms for an area of the location based on weather information associated with the location;
calculate a predictive yield for at least some of the plurality of germplasms for the area based on the respective RM for the at least some of the plurality of germplasms; and
generate the germplasm information for the at least some of the plurality of germplasms indicative of a respective performance for the area of the location based on the respective predictive yield.

22. The non-transitory computer-readable medium of claim 21, wherein the instructions for, calculating the predictive yield, cause the control circuitry to calculate the predictive yield based on at least one of year to year variance data associated with the germplasm, a product ranking of the germplasm, or penalty data associated with the germplasm.

23. The non-transitory computer-readable medium of claim 21, wherein the instructions cause the control circuitry to further determine penalty data associated with each of the at least some of the plurality of germplasms based on historical moisture data, wherein calculating the predictive yield comprises calculating the predictive yield further based on the penalty data.

24. The non-transitory computer-readable medium of claim 21, wherein the instructions for, when calculating the RM of each of the germplasms, cause the control circuitry to:
determine, for each acre of the area of the location, an aggregate Growth Degree Days (GDD) value based on historical weather information associated with the location;
modify the aggregate GDD value by one or more statistical operations; and
calculate an RM acreage indicative of the number of acres in the area projected to achieve relative maturity based on the modified aggregate GDD value.

25. The non-transitory computer-readable medium of claim 21, wherein the instructions for, calculating the RM acreage, cause the control circuitry to calculate the RM acreage based on a weather volatility value predictive of a likelihood of weather prediction error.

26. The non-transitory computer-readable medium of claim 21, wherein the germplasm information comprises a plurality of scenarios, and the instructions cause the control circuitry to further present an ordered list of the plurality of scenarios in order of respective performance.

27. The non-transitory computer-readable medium of claim 21, wherein the calculation of at least one of the RM or the predictive yield are implemented via control circuitry using a machine learning model.

28. The non-transitory computer-readable medium of claim 27, wherein the machine learning model comprises at least one of: a neural network, a deep neural network, a convolutional neural network, or a generative adversarial network.

29. The non-transitory computer-readable medium of claim 22, wherein the instructions for, calculating the predictive yield, cause the control circuitry to further calculate the predictive yield based on at least one of year to year disease variance data associated with germplasm.

30. The non-transitory computer-readable medium of claim 29, wherein the disease variance data associated with germplasm is based on locational data.
